(19) United States
(12) Patent Application Publication
(10) Pub. No.: US 2026/0000822 A1
(43) Pub. Date: Jan. 1, 2026

(54) BLOOD CONTAINER FOR COLLECTING AND REUSING WOUND BLOOD DURING OPERATIONS

(71) Applicant: Ardian HALILI, Schweiz (CH)

(72) Inventor: Ardian HALILI, Schweiz (CH)

(21) Appl. No.: 19/120,788

(22) PCT Filed: Sep. 13, 2023
(86) PCT No.: PCT/EP2023/075176

§ 371 Date: Apr. 13, 2025

(30) Foreign Application Priority Data
Oct. 13, 2022 (CH) CH001209/2022

(51) Int. Cl. A61M 1/00; A61J 1/05

(52) U.S. Cl. CPC A61M 1/60; A61J 1/05

(57) ABSTRACT

A blood collection container is fitted at the top with an intake connector for connecting a drainage tube and fitted with a suction connector for connecting the suction tube of a vacuum connection. The blood collection container also has a discharge port at the bottom for draining collected blood via a tube to a blood-cleaning apparatus contains an anticoagulant, and is sterilized as a whole and packaged in a delivery bag, which is sealed in a sterile manner by a hermetic seal to be opened in the operating theatre before use. A tube portion is advantageously placed on the lower drain port, which tube portion can be closed by a tube clamp, in order to connect a drain tube to this drain port and optionally opening or closing the tube portion or drain tube by the tube clamp.

### TECHNICAL FIELD

The present invention relates to a blood container for collecting the patient's blood in the event of unexpected blood loss during an operation in an operating theater and for—after cleaning with machine autotransfusion MAT, for example in a so-called Cell Saver®, to reuse it immediately.

### BACKGROUND

Blood loss occurs during all invasive operations. Such blood has so far been together with the sodium chloride which is being used for continuing wound cleansing sucked into a bag, for which there are vacuum lines or vacuum line connections in the operating theaters. A tube leads from the vacuum connection into the bag, and another tube, a drainage tube with a suction opening, leads from the operating table to the bag and there near the nozzle for the vacuum line into the bag.

During the operation, when the patient is bleeding, the wound is continuously washed with sodium chloride and the escaping blood, together with the sodium chloride used, is continuously suctioned off in the suction line due to the air flow generated, similar to the saliva at the dentist's, and reaches the collection bag via this tube. After the operation, this blood bag is disposed of. The blood is lost.

Only if the operation indicates that a high blood loss from the outset of more than 500 ml is to be expected a blood purification device is placed in the operating theater, a so-called Cell Saver®. These operations include heart and vascular surgery, abdominal surgery and transplant surgery, orthopaedic and trauma surgery and, with restrictions, tumor surgery and obstetrics. Machine autotransfusion MAT using Cell saver® helps to minimize the need for foreign blood and the associated risks. The costs per use of a Cell saver® are between CHF 180 and CHF 250 per operation.

If a Cell Saver® is used, the aspirated blood is fed directly via the drainage tube into the Cell Saver®, where any solids present in the blood are filtered out and the blood is being purified. All systems on the market work according to the centrifuge principle. Once the wound or drainage blood has been collected, it must not simply be returned to the patient without being processed. The reasons for this include the risk of coagulation activation and the risk of flooding the patient with cytokines, endotoxins and other biologically active substances. The blood is therefore filtered and centrifuged in the processing device. In particular, the following components are removed:
Activated and non-activated coagulation factors
Complementary factors
Free hemoglobin
Thrombocytes
Leukocytes (depending on the system to to 99% reduction)
Heparin
Antibiotics
Fat (up to 99.8%, depending on the system)
Inflammatory mediators

In return, the erythrocytes are enriched. The processed blood is fed via a delivery tube into a hanging blood bag and from there it is infused into the patient. This blood then contains 25,000 IU of an anticoagulant, for example Heparin® or sodium citrate, per 500 ml volume. 25,000 IU are sufficient for up to 3 liters of blood.

If a patient suffers a high blood loss during an operation that is not expected, there is regularly no Cell Saver® available in the operating theater. As a result, the blood is sucked into a conventional blood collection bag which can hold up to 3 liters. However, this blood cannot be reused as it may be contaminated with particles and some of it has already coagulated.

This blood is lost and must be disposed of, which is why, in the event of unexpectedly high blood loss, it has so far been necessary to use foreign blood which is usually available within around 30 minutes. However, this foreign blood must first be tested—it must belong to the correct blood group and it must also be checked that the patient in his blood does not have any antibodies that would make it incompatible for the patient. If the test is successful, the foreign blood can be administered to the patient via an infusion. Until it is actually available for an infusion, it takes about 30 minutes from the time it is ordered within the hospital, and up to a maximum of 3 hours. In cases where a patient's aorta is accidentally cut, mass infusions are required, approximately 20 to 40 standard blood bags.

The problems of current blood management can be illustrated using practical examples: During the routine insertion of hip prostheses, blood losses of 200 to 700 ml have been observed in practice. A person has approximately 70 ml of blood per kg of body weight. If a lightweight person weighs 45 kg, for example, they have around 3150 ml of blood in them, and if they then loses 1000 ml of blood, this becomes critical. With so little blood the person can absorb correspondingly less oxygen. The risk of infection increases and if this happens that a patient must be kept in the hospital for an extra day, with the corresponding cost consequences. In general, blood washing is worthwhile from a blood loss of approx. 500 ml. It would be much simpler and more effective if wound blood could be used in such cases of unexpectedly high blood loss. However, for this, operating theaters and their equipment are not yet equipped. Unfortunately, the unexpectedly leaking blood is regularly lost.

In view of the facts described above, it is the task of the present invention to make the use of external blood as dispensable as possible in principle, especially in cases of unexpectedly high blood loss, i.e. when there is no blood purification device or no Cell Saver® is available in the operating room. Rather, according to the invention, the wound blood should nevertheless still be able to be reused in such a case.

This problem is solved by a blood collection container with a suction nozzle at the top for connecting a drainage hose and a suction nozzle for connecting the suction hose of a vacuum connection, which is characterized in that the blood collection container has a discharge nozzle at the bottom for discharging collected blood via a hose to a blood purification device, and further, that the blood collection container contains an agent for inhibiting blood coagulation and is sterilized as a whole and is packed in a delivery bag which is sterilely sealed by means of a hermetic closure, for opening in the operating room shortly before for use.

### BRIEF DESCRIPTION OF THE FIGURES

The container is shown and as an example embodiment in the figures described below.

It shows:

FIG. 1: A conventional blood collection container in the form of a bottle;

FIG. 2: The blood collection container according to the invention, packed in a delivery bag;

FIG. 3: The blood collection container seen from above;

FIG. 4: The lower end of the unpacked blood collection container with the specially positioned spout with hose clamp for draining blood into a Cell Safer®;

FIG. 5: The suspended blood collection container in use in an operating theatre, with a supply tube for the drainage blood at the top, an air suction tube to a vacuum connection and a drain tube at the bottom for supplying blood to a cell saver.

### DETAILED DESCRIPTION

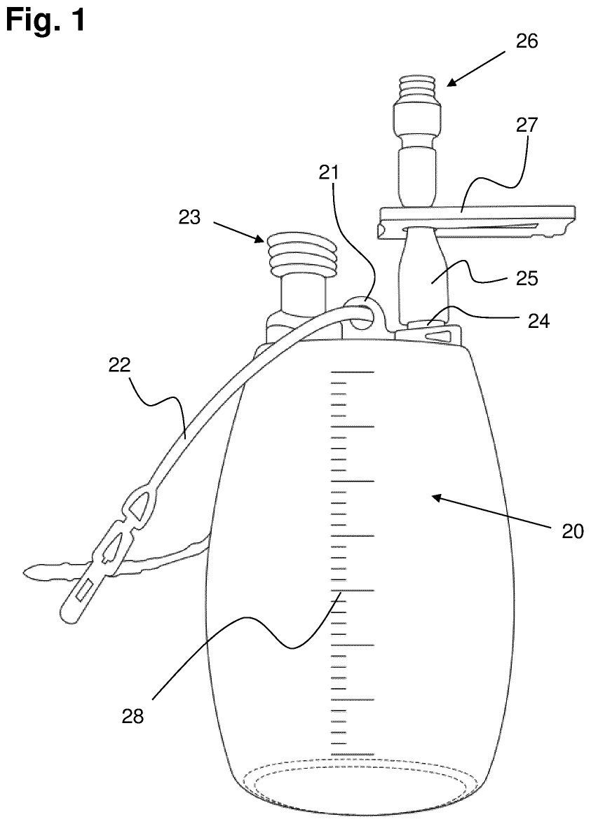

FIG. 1 first shows a conventional blood collection container in the form of a collection bottle plastic 20. It has a tab 21 at the top and an associated plastic binding strip 22 so that it can easily hunged on a tand or hook next to the operating table. On the top, two nozzles 23, 24 open outwards. One nozzle 23 is used to connect a vacuum hose from a stationary vacuum connection in the operating room, the other nozzle 24 is used to connect a drainage hose, which has a drainage suction nozzle at the other end, for suctioning wound blood from bleeding areas of the patient, i.e. where the patient has a wound or his body has been opened for surgical purposes. A hose section 25 is slipped over this nozzle 24 which forms again a nozzle 26 at its upper end, to which the drainage hose can be connected. The hose section 25 is equipped with a hose clamp 27 for, when the bottle is full and needs to be changed, to temporarily prevent the inflow of blood. The wounds or openings are continuously cleaned with sodium chloride NaCl, which is then mixed with the blood and fed into this collection bottle 20. In the example shown, a measuring scale 28 is attached to the bottle so that volume of the contents can be read at any time. When such a bottle 20 is full, it is replaced by an empty bottle, but the full bottle and in particular the wound blood collected in it must be disposed of and is definitely lost, even though it is valuable wound blood. In an emergency, if the patient requires additional blood due to severe blood loss, it is standard practice to use blood from another source. This is available in a hospital within 30 minutes to a maximum of 3 hours. However, it must first be checked. The blood group must match and this foreign blood must not have any antibodies which would else cause an incompatibility with the patient's blood.

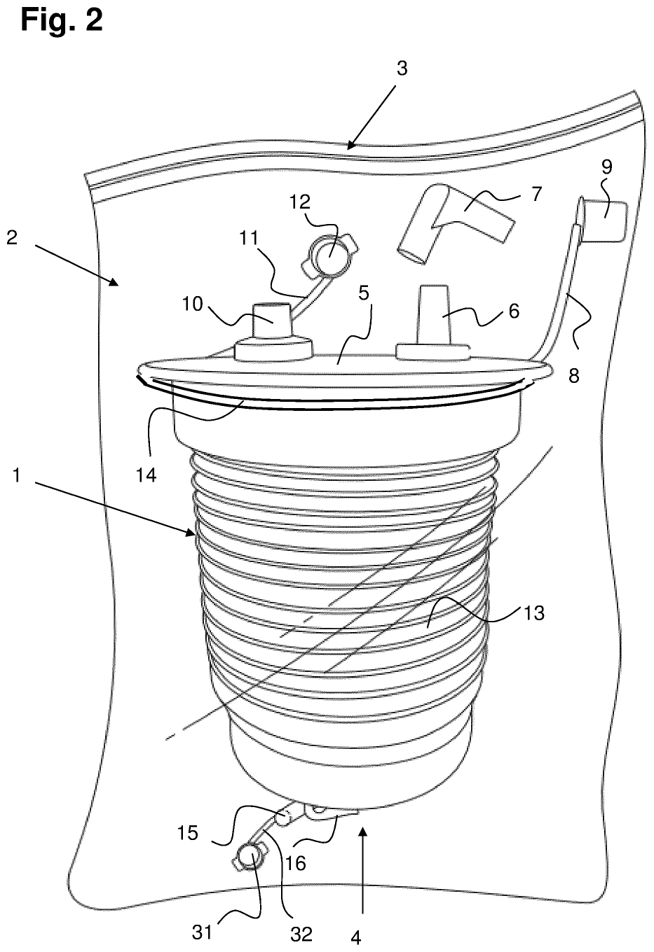

FIG. 2 shows a blood collection container 1, here in the form of a bellows-type bag according to the present invention, here sterile and sealed in a plastic bag 2 with a hermetically sealable closure 3, at the top, i.e. with an airtight and watertight closure 3. As a special feature, this blood collection bag 1 has a further nozzle 4 at its lower end for connecting a blood drainage tube. Here you can see a piece of a auxiliary tube 15, which is equipped with a tube clamp 16. This auxiliary tube 15 also includes a cap 31 on a securing band 32, so that the auxiliary tube 15 or the blood discharge tube to be connected to the nozzle 4 instead of the auxiliary tube 15 can be sterilely sealed at the end with this cap 31. This cap 31 with its securing band can also be formed onto the nozzle 4. In general, the bag can contain a few more sterile reserve caps so that it can always be closed sterilely in order to be able to safely store a patient's wound blood for a period of time. On the upper side of the blood collection container 1, here formed by a dimensionally stable plate-shaped lid 5, two slightly conical nozzles 6, 10 are attached towards their free end or are in one piece formed out of this lid 5 in molded plastic. On both sides of the lid 5, suspension brackets 14 are integrally molded with their two ends, whereby these two suspension brackets 14 can be swung up towards each other, after which the container 1 can be hung with horizontally oriented lid 5 on a stand or hook beside the operational table. One of the nozzles 6 on the lid can optionally be fitted with an elbow 7, to the other end of which a hose can then be attached in order to extract air via this nozzle 6 when the hose is connected to a stationary vacuum connection in the operating room. The attached elbow 7 ensures that the suction hose, when the container is suspended and the suction hose is connected to the vacuum connection on a wall, will not be kinked, what else would restrict or interrupt the air flow. The connection piece 6 or the attached angle piece 7 can be sealed by a cap 9. To prevent the cap 9 from being lost, it is attached with a safety strap at the free end directly to the cover 5. The second connection piece 10 in the lid 5 is used to connect a drainage tube in order to suck blood and sodium chloride 8 from wounds or open areas of a patient's body. Also this nozzle 10 is held 11 by an associated cap 12 on a safety strap and can be closed tightly by this cap 12. The plastic bag of the container, which is tightly connected to at the bottom, is designed as a bellows 13 so that it can grow downwards as the amount increases of blood until it can hold a maximum of 3 liters of blood volume. Designed as bellows 13, it takes up fewer space than a rigid bottle when not in use or for transportation or storage.

According to the invention, this bellows 13—and this is very important—is from filled from the very beginning with an agent for inhibiting blood coagulation in a sufficient quantity. For every 500 ml of blood volume, for example, the blood can contain at least 25,000 IU of an anticoagulant called Heparin® in order to safely prevent the blood from coagulation. 25,000 IU in case of Heparin® are sufficient for up to 3 liters of blood. Even if less than 500 ml of blood ends up in the container 1, there are no disadvantages associated with this, even if it contains 25,000 IU of this anticoagulant. Alternative anticoagulants, for example sodium citrate or others are also suitable. This blood collection container 1 is therefore delivered from the factory in the condition shown here in FIG. 2 and stored temporarily in the hospital until it is needed. Its important features are, firstly, that it has a connection piece at its lower end 4 for connecting a blood drainage tube with a tube clamp 16. Secondly, it already contains an anticoagulant in sufficient quantity for the full volume of blood that container 1 can hold, for example up to 3 liters of blood. Thirdly, the entire container 1 has been sterilized before it was a hermetically packed in a delivery bag 2 at the factory. And it is packed into the delivery bag 2 under sterile conditions and temporarily stored in this delivery bag 2 in the hospital. Whenever the blood collection container 1 is used, it is removed from the delivery bag 2 by opening the hermetically sealed closure 3 and hung on a stand in the operating theater. The blood removal tube is then connected to the lower nozzle 4 and this tube is either closed with a tube clamp 16 or directly connected to a Cell Saver®.

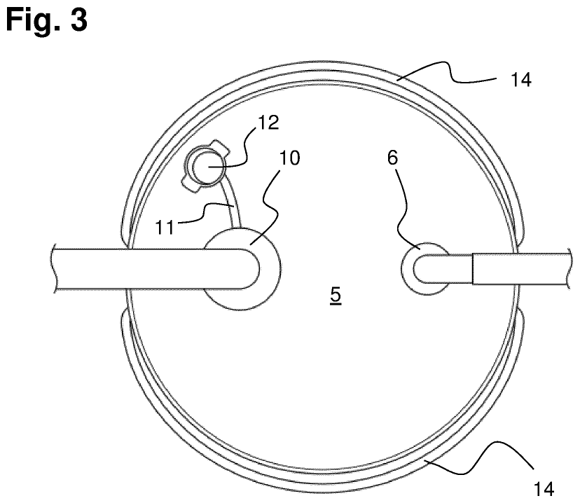

FIG. 3 shows a view of the lid of the blood collection container seen from above. The two nozzles 6, 10 as well as the connected tubes and a cap 12 with safety strap 11 can be seen. On both sides of the circumference, integrally formed brackets 14 are formed on the lid 5, which are shown in the resting position. If necessary, they can be swiveled up by 90° and then form two support brackets for hanging the the blood collection container on a hook.

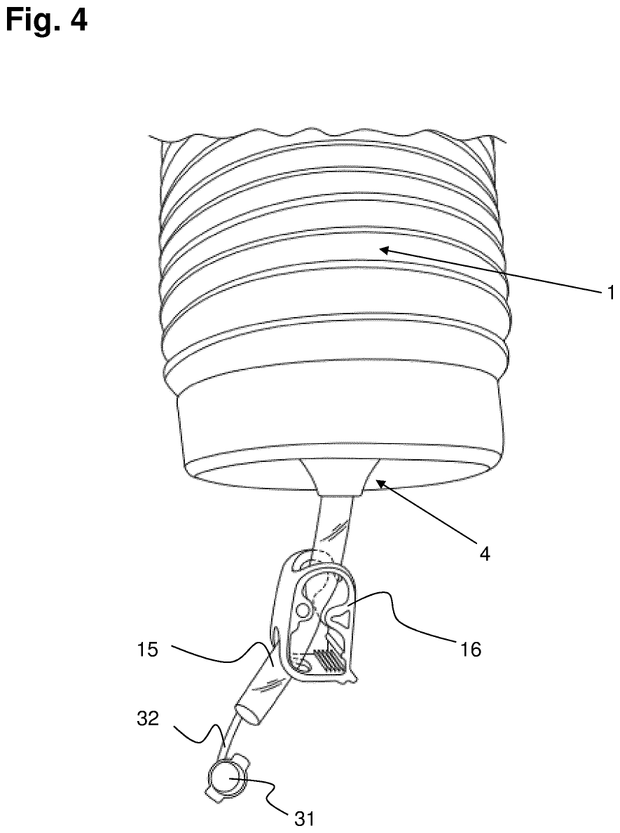

FIG. 4 shows the lower end of the blood collection container according to the invention 1. Here one an see the discharge nozzle 4 and an auxiliary hose 15 attached to it with a hose clamp 16. There is also a sterile cover cap 31, which is attached to a safety strap 32 so that it is always available to cover the auxiliary tube 15 or a blood drainage tube 30 to be connected and closed in a sterile manner. To connect the effective blood drainage tube, this small tube section 15 is removed and the tube clamp 16 will be pushed onto the effective blood draining tube and then the clamp 16 is either closed or opened for a planned direct blood drainage.

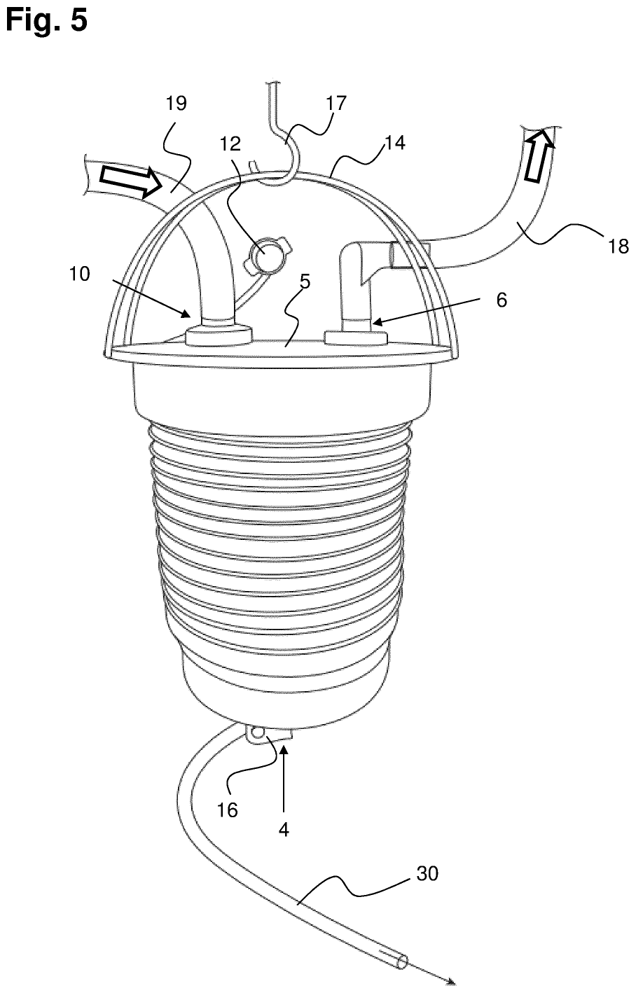

FIG. 5 shows this blood collection container 1 according to the invention in practical use. It is suspended by its two suspension brackets 14 from a hook 17. At the top, the elbow 7 and a suction hose 18 are connected to its suction nozzle 6, which leads to a vacuum connection, as present in every operating theater.

The blood drainage tube 19 is connected to the other nozzle 10. At the bottom of the bellows 13 of the blood collection container 1, the blood drainage hose 30 is connected to the drainage nozzle 4 there, as is the hose clamp 16. As in the suction hose shown with arrows hose 19, air is sucked out of the container via the suction hose 18 and the induced air flow into the drainage hose 18 and drainage continues 19, so that with its free end and blood can be conveniently sucked out of wounds or open patient sites a suction piece attached there, according to the same principle as saliva is sucked out of the patient's mouth at the dentist, for example.

If the tube clamp 16 in the blood drainage tube is closed, blood will continuously enter container 1 by the sucking, together with sodium chloride, which is used for ongoing wound cleansing. The container 1 will be gradually filled up. When the container 1 is full, the tubes 18, 19 can be removed at the top and the nozzles 6, 10 can be closed with their caps 9, 12. At the bottom, the container 1 is still sealed by the hose clamp 16. The blood collected from the patient is precious wound blood and is now available for transfusion. It does not clot and is kept steril. If complications arise during an operation or if un expectedly high blood loss occurs, this collected wound blood can be transfused directly thanks to this container 1 as soon as a Cell Saver® is placed in the operating room or is already there. In the event that such a blood purification device or a Cell Saver® must be brought into operating room, this has of course to be done in sterile conditions, i.e. only a previously sterilized blood device may be brought into the operating room. In this case, the blood draining tube 30 will be connected to the lower nozzle 4 and its other end will be connected to the Cell Saver® so that the collected wound blood in the Cell Savercan be cleaned and then fed directly into an infusion bag, from where it can be administered to the patient via an infusion. The use of foreign blood and the associated logistical and medical measures can be avoided. This saves time and money and prevents infections. In addition, patients who have suffered relatively high blood loss can be discharged from hospital earlier, usually one or even several days earlier than usual. This in turn saves considerable costs.

Wound blood is the most problem-free blood for the patient anyway, and thanks to this blood collection container 1 it is no longer as lost before, but is available to the patient immediately or even with interruptions of several hours or days at any time. This means that a patient will never bleed to death on the operating table again.

1-6. (canceled)

7. A blood collection container comprising:
a suction nozzle at the top for connecting a drainage hose and a suction nozzle for connecting the suction hose of a vacuum connection;
a discharge nozzle at the bottom for discharging collected wound blood via a blood discharge hose to a blood purification device; and
an agent for inhibiting blood coagulation in a quantity for a volume of blood which the container can hold, wherein the agent is sterilised as a whole and packed in a delivery bag that is sterilely sealed by a hermetic closure for opening in an operating theatre before use.

8. The blood collection container of claim 7, wherein a hose section is fitted to the lower discharge nozzle as an auxiliary hose for connecting the blood discharge hose to this discharge nozzle, wherein the hose section can be closed with a hose clamp, so that by means of the hose clamp the hose section or the blood discharge hose to be connected can be selectively opened or closed, and wherein a closure cap can be fitted to the discharge nozzle or to the hose section via a securing means. the blood discharge hose to be connected can be optionally opened or closed by means of the hose clamp, and wherein a sealing cap is moulded onto the discharge nozzle or the hose section via a securing band.

9. The blood collection container of claim 7, wherein the container is designed as a bellows so that in the suspended state its volume can expand downwards with the amount of blood supplied and it can grow downwards to its full volume.

10. The blood collection container of claim 7, wherein the container is sealed at the top with a disc-shaped lid, wherein the lid is perforated by the two nozzles for connecting the suction hose or the drainage hose.

11. The blood collection container of claim 10, wherein the nozzles can each be sealed by a cap, which are each held on the lid by a securing band.

12. The blood collection container of claim 10, wherein it has two suspension brackets moulded onto its upper lid, which can be swivelled up towards each other, for suspending the blood collection container from a hook.

13. The blood collection container of claim 7, wherein an angle piece can be placed on the connecting piece to avoid kinks in the outgoing or incoming vacuum or drainage hose.

14. The blood collection container of claim 7, wherein the volume of blood that the container can hold comprises 3 litres of blood.
